(19) United States
(12) Plant Patent Application Publication
(10) Pub. No.: US 2007/0163022 P1
(43) Pub. Date: Jul. 12, 2007

(54) New Guinea Impatiens plant named 'Fisnics Salmgold'

(76) Inventor: Birgit Christa Hofmann, Bendorf (DE)

Correspondence Address: JONDLE & ASSOCIATES P.C., 858 HAPPY CANYON ROAD SUITE 230, CASTLE ROCK, CO, 80108, US

(21) Appl. No.: 11/330,445

(22) Filed: Jan. 12, 2006

(51) Int. Cl. A01H 5/00

(52) U.S. Cl. PLT/318

(57) ABSTRACT

A new New Guinea Impatiens plant particularly distinguished by large, round to oval, flat, orange flowers with a white center that are borne well above the foliage, early to medium flowering, deep green foliage with yellow variegation, and a medium to tall plant habit that is round and bushy, is disclosed.

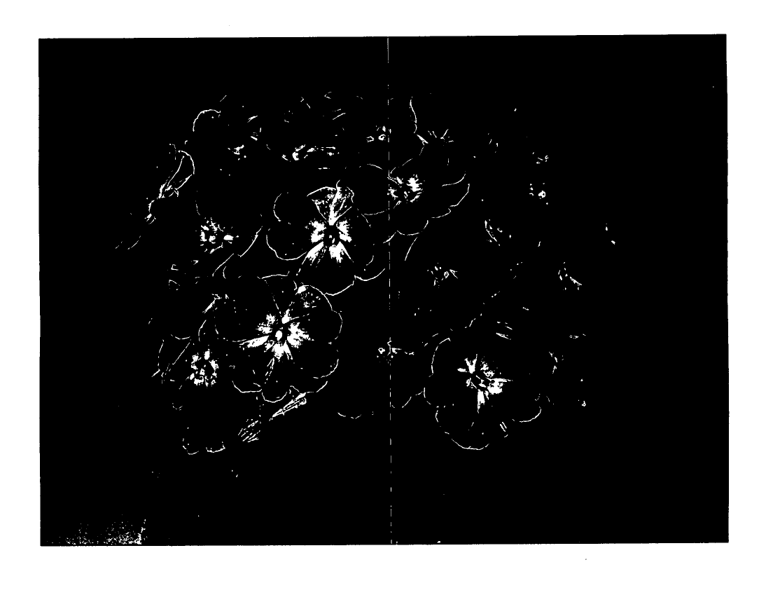

### GENUS AND SPECIES

Impatiens hawkeri W. Bull

### VARIETY DENOMINATION

‘Fisnics Salmgold’

### BACKGROUND OF THE NEW PLANT

The present invention comprises a new and distinct cultivar of New Guinea Impatiens, botanically known as Impatiens hawkeri W. Bull, and hereinafter referred to by the cultivar name ‘Fisnics Salmgold’. The new cultivar originated from a hybridization made in the year 2001 in Hillscheid, Germany. The female parent was the proprietary New Guinea Impatiens plant ‘K01-8565-1’ (unpatented), while the male parent was the proprietary New Guinea Impatiens plant ‘K01-8132-7’ (unpatented). The seeds produced by the hybridization were sown in Galdar, Gran Canaria, Spain, in November 2001. A single plant selection was chosen for further evaluation and for asexual propagation in April 2002 in Galdar, Gran Canaria, Spain.

The new cultivar was created in 2001 in Hillscheid, Germany and has been asexually reproduced repeatedly by vegetative cuttings and tissue culture in Galdar, Gran Canaria, Spain and Hillscheid, Germany over a two-year period. ‘Fisnics Salmgold’ has not been observed under all possible environmental conditions. The present invention has been found to retain its distinctive characteristics through successive asexual propagations.

Plant Breeder's Rights for this cultivar were applied for in Switzerland on Oct. 13, 2004 and in Canada on Apr. 20, 2005.

### SUMMARY OF THE INVENTION

The following are the most outstanding and distinguishing characteristics of this new cultivar when grown under normal horticultural practices in Hillscheid, Germany and Langley, British Columbia, Canada.
1. Round to oval, flat flowers of orange color with a white center;
2. Very large flowers borne well above the foliage;
3. Deep green foliage with yellow variegation;
4. A medium to tall, round and bushy plant habit;
5. An early to medium flowering response; and
6. Floriferous throughout the summer.

### DESCRIPTION OF PHOTOGRAPH

This new New Guinea Impatiens plant is illustrated by the accompanying photograph which shows overall plant habit including blooms, buds, and foliage of the plant; the colors shown are as true as can be reasonably obtained by conventional photographic procedures. The photograph is of a whole plant about 14-weeks old, grown in a greenhouse in Hillscheid, Germany, in late Spring (late May).

### DESCRIPTION OF THE NEW CULTIVAR

The following detailed descriptions set forth the distinctive characteristics of ‘Fisnics Salmgold’. The data which define these characteristics were collected from asexual reproductions carried out in Hillscheid, Germany. The plant history was taken on 13- to 14-week old plants which were planted as rooted cuttings in 12-cm pots in late February 2004, and then grown in a greenhouse at a minimum temperature of 16° C. Color readings were taken under natural light in the greenhouse. Color references are primarily to the RHS Colour Chart of The Royal Horticultural Society of London (RHS) (2001).

### DETAILED BOTANICAL DESCRIPTION OF THE NEW PLANT

Classification:
   Family.—Balsaminaceae.
   Botanical.—Impatiens hawkeri W. Bull.
   Common name.—New Guinea Impatiens.
Parentage:
   Female parent.—‘K01-8565-1’ a proprietary salmon and white flowered New Guinea Impatiens plant (unpatented).
   Male parent.—‘K01-8132-7’ a proprietary pale-violet flowered New Guinea Impatiens plant (unpatented).
Growth:
   Growth and branching habit.—Medium to large size, round, bushy, and well-branched; the growth is indeterminate, though weak after the beginning of flowering.
   Height.—15.2 cm.
   Width.—29.5 cm.
   Propagation.—Terminal tips for cuttings.
   Time to produce a finished flowering plant.—9 to 10 weeks for a 12-cm pot.
   Time to initiate and develop roots.—18 days at 22° C.
Branches:
   Average number.—8 to 10.
   Length of branches.—12 cm.
   Internode length.—2.5 cm to 4.5 cm.
   Diameter of branches.—0.5 cm to 0.7 cm.
   Stem color.—RHS 185B (purple-brown).
Leaves:
   Arrangement.—Primarily in whorls.
   Size.—Length: 11.9 cm Width: 3.7 cm.
   Shape.—Elliptic, relatively narrow.
   Margin.—Slightly serrate, ciliated.
   Apex.—Acuminate.
   Base.—Acute.
   Color (mature leaves).—Upper surface: Between RHS 137A and RHS 139A (intense green) Lower surface: RHS 139C (light-green).
   Color (immature leaves).—near RHS 137D.
   Texture.—Surface is slightly glossy and faintly rippled.
   Venation color.—Upper surface: RHS 49D (pale light-pink) Lower surface: RHS 47C (dull light-red)
   Variegation.—Upper surface: RHS 13B (yellow stripe) the yellow area may cover up to ⅔ of the leaf's width, depending on the light intensity Lower surface: RHS 13C (yellow).
   Petioles.—Length: 1.0 cm Diameter: 0.3 cm to 0.4 cm Color: Upper surface: RHS 47C Lower surface: RHS 47B Texture: Glabrous.
Flower buds:
   Shape.—Ovoid.
   Size.—Length: 2.5 cm Diameter: 1.8 cm.
   Color at tight bud.—RHS 33A.
Inflorescence:
   Blooming habit.—9 weeks after planting of rooted cuttings
   Inflorescence type.—Flowers appear solitary in the leaf axils of the upper nodes of the stems.
   Number of flowers per node.—6 to 8, in various stages of development
   Flowering season.—Continuously, mainly from March to October, depending on light intensity.
   Lastingness of individual blooms.—Up to 12 days (in a greenhouse in the Spring at 18° C.)
   Fragrance.—None.
   Peduncle.—Color: RHS 47A (dull red) Size: Length: 6.5 cm Diameter: 0.15 cm Texture: Flexible, glabrous.
Flowers:
   Form of corolla.—Single-type, 5 petals.
   Shape of corolla.—Nearly round to oval, with the measured length diameter about 1 cm shorter than the width, with the petals overlapping, almost flat.
   Corolla size.—Length: 8.0 cm Width: 8.9 cm Depth: 1.0 cm.
Petals:
   Shape.—Cordate, weakly lobed at the top ends.
   Apex.—Emarginate.
   Base.—Attenuate.
   Margin.—Entire.
   Petal size.—Top: Length: 3.4 cm Width: 5.4 cm Lateral: Length: 4.0 cm Width: 4.3 cm Lower: Length: 4.0 cm Width: 4.8 cm
   Color.—General color description: Bright orange with a white center Upper surface: RHS 40A to RHS 40B (bright-orange), with RHS N155D (white) near the bases of the petals Lower surface: RHS 40C to RHS 40D Eye Zone: RHS 57B (purple, small) Petal texture: Smooth, satiny Aspect: Flat, parallel to the surface of the foliage.
Spur:
   Shape.—Downwardly curved.
   Color.—RHS 45D (light-red).
   Size.—Length: 6.0 cm Diameter: 0.3 cm (at the flower end).
Reproductive organs:
   Stamens.—Number: 5 fused Stamen color (upper surface color): RHS 46D Anthers: Fused, hooded Pollen color: RHS 8D (whitish-yellow).
   Pistils.—Style and stigma: 5, very short, RHS 8D (pale-yellow) Ovary: 5-celled Ovary length: 0.6 cm Ovary color: RHS 141C (surface green).
Fruit and Seed Set: No seed set observed.
Disease and insect resistance: No particular resistance or susceptibility has been observed.

### COMPARISON WITH PARENTAL AND COMMERCIAL CULTIVARS

‘Fisnics Salmgold’ differs from the female parent ‘K01-8565-1’ (unpatented), by having a deeper orange flower color, and variegated foliage, while ‘K01-8565-1’ has salmon and white flowers and uniform, medium-green leaves.

‘Fisnics Salmgold’ differs from the male parent ‘K01-8132-7’ (unpatented), by having orange colored flowers and variegated foliage while ‘K01-8132-7’ has large, pale violet flowers and uniform green foliage.

‘Fisnics Salmgold’ differs from the commercial cultivar ‘Fisnics Salmon Ice’ (U.S. Plant Pat. No. 14,812) by having a deeper orange main flower color, larger flowers, larger leaves, and generally taller plant habit as well as variegated foliage with distinct yellow stripes, while ‘Fisnics Salmon Ice’ has uniform, green foliage.

‘Fisnics Salmgold’ differs from cultivar ‘Fisupnic Salmice’ (U.S. Plant Pat. applied for) by having orange colored flowers with a less pink hue and a more distinct purple eye. Additionally, ‘Fisnics Salmgold’ has variegated green and yellow foliage, while the leaves of ‘Fisupnic Salmice’ are uniform in color.

‘Fisnics Salmgold’ differs from the commercial cultivar ‘Fisnics Dark Salmon’ (U.S. Plant Pat. No. 13,232) by having a white eye in the center. Additionally, the foliage of ‘Fisnics Salmgold’ is a lighter green color and shows a yellow stripe along the midrib, while the foliage of ‘Fisnics Dark Salmon’ is a uniform dark green.

I claim:

1. A new and distinct cultivar of New Guinea Impatiens plant as shown and described herein.
